(19) United States
(12) Patent Application Publication
(10) Pub. No.: US 2006/0173337 A1
(43) Pub. Date: Aug. 3, 2006

(54) Intravascular ultrasound probing device

(76) Inventors: Yu Shih Chen, Taoyuan City (TW); Wen Pin Hsieh, Sanwan Township (TW); Ih Yuan Kuo, Shengang Township (TW); Pao Hung Lin, Changhua City (TW)

Correspondence Address: VOLENTINE FRANCOS, P.L.L.C., Suite 150, 12200 Sunrise Valley Drive, Reston, VA, 20191, US

(21) Appl. No.: 11/155,612

(22) Filed: Jun. 20, 2005

(30) Foreign Application Priority Data
Dec. 29, 2004 (TW) 093141103

(51) Int. Cl. A61B 8/06

(52) U.S. Cl. 600/454

(57) ABSTRACT

The present invention provides an intravascular ultrasound probing device using Doppler effect. The intravascular ultrasound probing device comprises a first tube, a first ultrasound probe head positioned in the first tube, a second ultrasound probe head positioned in the first tube and an separating member positioned between the first ultrasound probe head and the second ultrasound probe head. Preferably, the first ultrasound probe head and the second ultrasound probe head are made of lead zirconate titanate (PZT), and can generate an ultrasound with a frequency between 1 MHz and 100 MHz. The first ultrasound probe head can measure the flow of the blood in a vessel, and the second ultrasound probe head can capture the image of the wall of the vessel. The separating member is preferably made of polymer for absorbing undesired reflection wave.

### BACKGROUND OF THE INVENTION

(A) Field of the Invention

The present invention relates to an intravascular ultrasound probing device, and more particularly, to an intravascular ultrasound probing device with Doppler effect.

(B) Description of the Related Art

Treatment of the intravascular diseases depends primarily on surgical operations. During the surgical operation, it is more important that instruments can help doctors to diagnose correctly. Therefore, an intravascular ultrasound system (IVUS) is widely used in the detecting apparatus for the intravascular disease operation.

U.S. Pat. No. 5,582,171 discloses an intravascular Doppler interference imaging system, which can acquire images around the wall of the blood vessel to assist in diagnosing the atherosclerosis, thrombus and intravascular obstruction. However, the intravascular Doppler interference imaging system disclosed in U.S. Pat. No. 5,582,171 cannot provide flow information of blood in the blood vessel, and therefore physicians cannot diagnose precisely.

### SUMMARY OF THE INVENTION

The objective of the present invention is to provide an intravascular ultrasound probing device with Doppler effect.

In order to achieve the above-mentioned objective and avoid the problems of the prior skills, the present invention provides an intravascular ultrasound probing device with Doppler effect. The intravascular ultrasound probing device comprises a first tube, a first ultrasound probe head positioned in the first tube, a second ultrasound probe head positioned in the first tube and an separating member positioned between the first ultrasound probe head and the second ultrasound probe head. Preferably, the first ultrasound probe head and the second ultrasound probe head are made of lead zirconate titanate (PZT), and can generate an ultrasound with a frequency between 1 MHz and 100 MHz. The first ultrasound probe head can measure the flow of the blood in a vessel, and the second ultrasound probe head can capture the image of the wall of the vessel. The separating member is preferably made of polymer for absorbing undesired reflection wave.

The present intravascular ultrasound probing device uses piezoelectric material to make the probe head, which is used not only to generate an ultrasound for probing, but also to receive the ultrasound reflected by the sample to be measured. In other words, the present intravascular ultrasound probing device uses the same element as a signal generator and signal receiver. Consequently, it is possible to integrate two probe heads inside a single tube to probe both the blood flow and the image of the wall of the blood vessel at the same time. The prior art uses the laser beam as the probing signal, which can only penetrate the organization for about 2 to 3 mm and the intensity of the probing signal attenuates dramatically. On the contrary, the present invention uses the ultrasound as the probing signal, which can penetrate the organization for about several centimeters and the intensity of the probing signal only attenuates slightly on penetrating the tube.

### BRIEF DESCRIPTION OF THE DRAWINGS

The objectives and advantages of the present invention will become apparent upon reading the following description and upon reference to the accompanying drawings in which:

FIG. 1 is a cross-sectional view of an intravascular ultrasound probing device according to the present invention; and

FIG. 2 and FIG. 3 are a signal flow diagram of the intravascular ultrasound probing device according to the present invention.

### DETAILED DESCRIPTION OF THE INVENTION

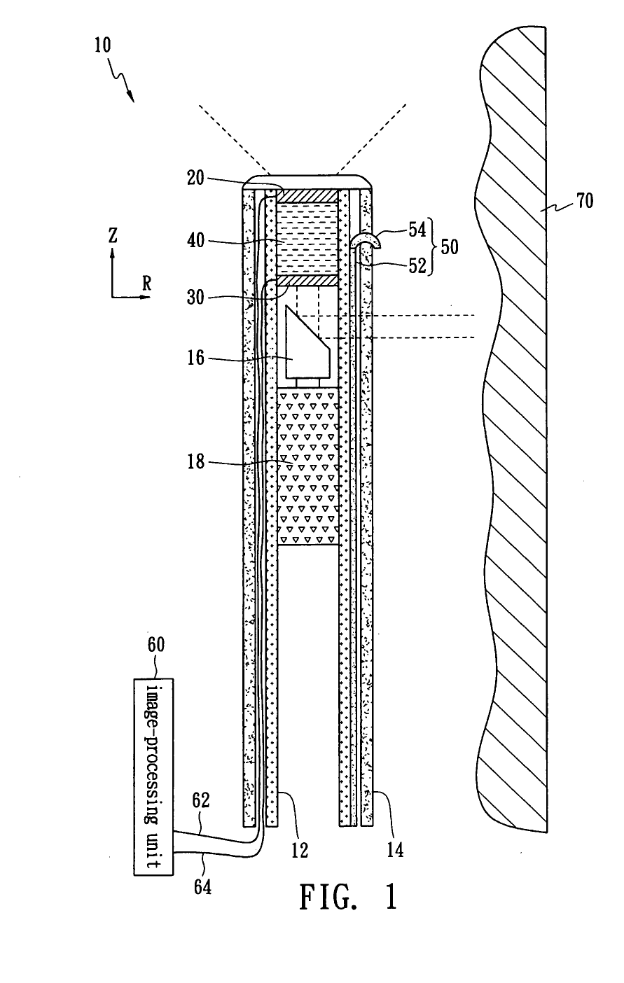

FIG. 1 is a cross-sectional view of an intravascular ultrasound probing device 10 according to the present invention. The intravascular ultrasound probing device 10 comprises a first tube 12, a first ultrasound probe head 20 positioned in the first tube 12, a second ultrasound probe head 30 positioned in the first tube 12 and a separating member 40 positioned between the first ultrasound probe head 20 and the second ultrasound probe head 30. Preferably, the first ultrasound probe head 20 and the second ultrasound probe head 30 are made of piezoelectric material such as lead zirconate titanate (PZT), and can generate an ultrasound with a frequency between 1 MHz and 100 MHz. The separating member 40 is preferably made of polymer to absorb unwanted reflecting ultrasound.

The intravascular ultrasound probing device 10 can further comprise a reflection mirror 16 positioned in the first tube 12, a driver 18 positioned in the first tube 12, a second tube 14, a guiding member 50 and an image-processing unit 60 (e.g. image processing circuit). The guiding member 50 comprises a body 52 positioned between the first tube 12 and the second tube 14, and barb 54 connected to the body 52 and protruding the second tube 14. When the intravascular ultrasound probing device 10 goes forward in a blood vessel of a human body and meets blood vessel branches, an operator can guide the intravascular ultrasound probing device 10 in the blood vessel via the guiding member 50 to any one of blood vessel branches.

The image-processing unit 60 is electrically connected to the first ultrasound probe head 20 and the second ultrasound probe head 30 via signal wires 62 and 64, respectively. The first tube 12 is positioned inside the second tube 14, i.e., the first tube 12 is an inner tube, while the second tube 14 is an outer tube, and a space between the inner tube and the outer tube is available for setting the signal wires 62 and 64.

The driver 18 can be a micromotor for rotating the reflection mirror 16 in order to guide the ultrasound from the second ultrasound probe head 30 to the wall of the blood vessel 70. In other words, the reflection mirror 16 will reflect the ultrasound from the second ultrasound probe head 30 from Z-axis to R-axis, while the driver 18 will rotate the reflection mirror 16 to allow the ultrasound from the second ultrasound probe head 30 to reach the wall of the blood vessel 70 at any angle.

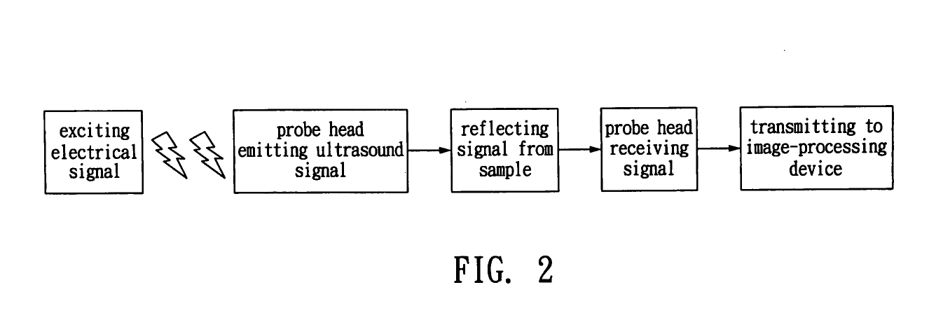

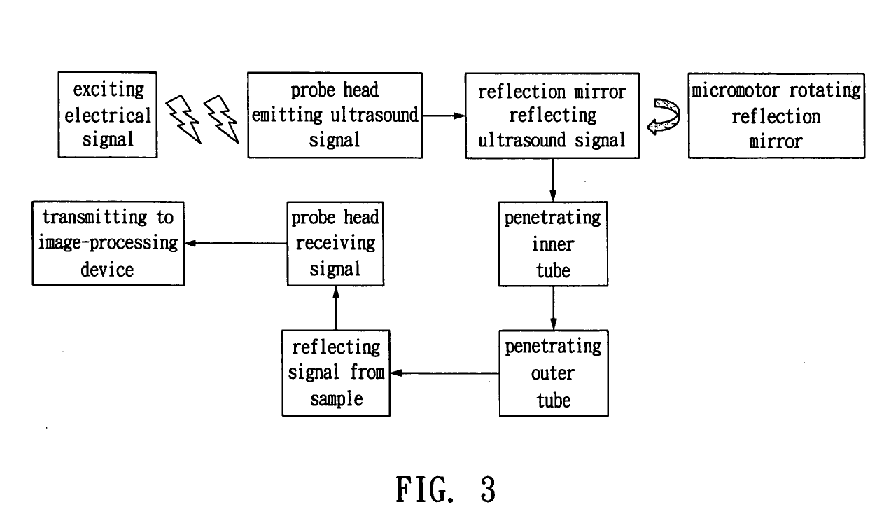

FIG. 2 and FIG. 3 are a signal flow diagram of the intravascular ultrasound probing device 10 according to the present invention. As shown in FIG. 2, an exciting electrical signal drives the first ultrasound probe head 20, made of PZT, to emit an ultrasound in the positive direction of Z-axis, which can penetrate an sample to be measured (e.g. blood in the blood vessel 70) to a predetermined depth (about several centimeters), and then reflected by the sample to the first ultrasound probe head 20. The first ultrasound probe head 20 receives the ultrasound reflected by the blood, and is then transmitted to the image-processing unit 60 via the signal wire 62. The image-processing unit 60 will predict the blood flow in the blood vessel 70 according to the ultrasound reflected by the blood in the blood vessel 70 to diagnose whether or not an intravascular obstruction occurs.

As shown in FIG. 3, an exciting electrical signal drives the second ultrasound probe head 30, also made of PZT, to emit an ultrasound at the negative direction of Z-axis, which is reflected by the reflection mirror 16 to R-axis to reach the wall of the blood vessel 70 after penetrating the first tube 12 and the second tube 14. The ultrasound can penetrate the wall of the blood vessel 70 to a predetermined depth and can then be reflected to the reflection mirror 16, which will further reflect the ultrasound to the second ultrasound probe head 30. The second ultrasound probe head 30 receives the ultrasound from the wall of the blood vessel 70 via the reflection mirror 16, and transmits the reflected ultrasound to the image-processing unit 60 via the signal wire 64. The image-processing unit 60 picks up the image of the wall according to the reflected ultrasound from the wall of the blood vessel 70. The driver 18 can rotate the reflection mirror 16 360° to pick up all images of the blood vessel 70.

Compared to prior art, the intravascular ultrasound probing device 10 according to the present invention possesses advantages listed in the table below:

| | Prior art | Present invention |
|---|---|---|
| Probe type | Optical | Ultrasound |
| Penetrating depth | Shallow: 2 to 3 mm | Deep: several centimeters |
| Operating frequency | ˜ | 1 MHz to 100 MHz |
| Reference end | Required | Not required |
| Exciting source | In the instrument | In the tube |
| Receiving end | In the instrument | In the tube |
| Efficiency | Attenuate dramatically | Attenuate on penetrating |
| | | the tube only |

As shown in the table above, the present intravascular ultrasound probing device 10 uses the probe head made of piezoelectric material, which is used not only to generate an ultrasound for probing, but also to receive the ultrasound reflected by the sample to be measured. In other words, the present intravascular ultrasound probing device 10 uses the same element as a signal generator and signal receiver. Consequently, it is possible to integrate two probe heads inside a single tube to probe both the blood flow and the image of the wall of the blood vessel at the same time. The prior art uses the laser beam as the probing signal, which can only penetrate the organization about 2 to 3 mm and the intensity of the probing signal attenuates dramatically. On the contrary, the present invention uses the ultrasound as the probing signal, which can penetrate the organization about several centimeters and the intensity of the probing signal only attenuates slightly on penetrating the tube.

The above-described embodiments of the present invention are intended to be illustrative only. Numerous alternative embodiments may be devised by those skilled in the art without departing from the scope of the following claims.

What is claimed is:

1. An intravascular ultrasound probing device, comprising:
a first tube;
a first ultrasound probe head positioned in the first tube for measuring the flow of the blood in a vessel;
a second ultrasound probe head positioned in the first tube for capturing the image of the wall of the vessel; and
a separating member positioned between the first ultrasound probe head and the second ultrasound probe head.

2. The intravascular ultrasound probing device of claim 1, wherein the first ultrasound probe head is made of lead zirconate titanate, and can generate an ultrasound with a frequency between 1 MHz and 100 MHz.

3. The intravascular ultrasound probing device of claim 1, wherein the second ultrasound probe head is made of lead zirconate titanate, and can generate an ultrasound with a frequency between 1 MHz and 100 MHz.

4. The intravascular ultrasound probing device of claim 1, further comprising:
a reflection mirror positioned in the first tube for guiding an ultrasound from the second ultrasound probe head to the wall of the vessel; and
a driver positioned in the first tube for rotating the reflection mirror.

5. The intravascular ultrasound probing device of claim 4, wherein the driver is a micromotor.

6. The intravascular ultrasound probing device of claim 1, further comprising a second tube, wherein the first tube is positioned in the second tube.

7. The intravascular ultrasound probing device of claim 6, further comprising a plurality of signal wires positioned between the first tube and the second tube for transmitting signals received by the first ultrasound probe head and the second ultrasound probe head.

8. The intravascular ultrasound probing device of claim 6, further comprising a guiding member positioned between the first tube and the second tube.

9. The intravascular ultrasound probing device of claim 8, wherein the guiding member comprises:
a body positioned between the first tube and the second tube; and
a barb connected to the body and protruding the second tube.

10. The intravascular ultrasound probing device of claim 1, further comprising an image-processing unit electrically connected to the first ultrasound probe head and the second ultrasound probe head.

11. The intravascular ultrasound probing device of claim 1, wherein the separating member is made of polymer.
